(19) United States
(12) Patent Application Publication
(10) Pub. No.: US 2024/0189328 A1
(43) Pub. Date: Jun. 13, 2024

(54) COMBINATION OF NOREPINEPHRINE REUPTAKE INHIBITOR AND A CANNABINOID FOR USE IN TREATING SLEEP APNEA

(71) Applicant: APNIMED, INC. (DELAWARE), Cambridge, MA (US)

(72) Inventors: Lawrence G. MILLER, Cambridge, MA (US); Luigi TARANTO-MONTEMURRO, Brookline, MA (US); Ronald FARKAS, Clarksville, MD (US); David P. WHITE, Denver, CO (US)

(21) Appl. No.: 18/286,399

(22) PCT Filed: Apr. 15, 2022
(86) PCT No.: PCT/US2022/024942

§ 371 Date: Oct. 11, 2023

Related U.S. Application Data
(60) Provisional application No. 63/175,641, filed on Apr. 16, 2021.

(51) Int. Cl. A61K 31/00; A61K 31/138; A61K 31/216; A61K 31/433; A61K 31/5375; A61P 11/04

(52) U.S. Cl. CPC A61K 31/658; A61K 31/138; A61K 31/216; A61K 31/433; A61K 31/5375; A61P 11/04

(57) ABSTRACT

Pharmaceutical compositions comprising a norepinephrine reuptake inhibitor (NRI) and a cannabinoid and optionally a muscarinic receptor antagonist (MRA) and/or carbonic anhydrase inhibitor (CAI) and methods of treating sleep apnea are described herein.

### CROSS-REFERENCE TO RELATED APPLICATION

This application claims priority to U.S. provisional application 63/175,641, filed Apr. 16, 2021, the entire contents of which are incorporated herein by reference.

### TECHNICAL FIELD

The present invention provides pharmaceutical compositions comprising (i) a norepinephrine reuptake inhibitor (NRI) and (ii) a cannabinoid, and optionally (iii) a muscarinic receptor antagonist (MRA) and/or carbonic anhydrase inhibitor (CAI), as well as methods of treating sleep apnea.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a norepinephrine reuptake inhibitor (NRI) and (ii) a cannabinoid.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the NRI is reboxetine or a pharmaceutically acceptable salt thereof. In some embodiments, the cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof. In some embodiments, the cannabinoid is CBD. In some embodiments, the cannabinoid is THC. In some embodiments, the cannabinoid is dronabinol, nabilone, or a combination thereof. In some embodiments, the cannabinoid is dronabinol. In some embodiments, the method further comprises administering to the subject (iii) a muscarinic receptor antagonist (MRA). In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the method further comprises administering to the subject a carbonic anhydrase inhibitor (CAI). In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the atomoxetine or pharmaceutically acceptable salt thereof is administered at a dose of from about 20 to about 200 mg. In some embodiments, the atomoxetine or pharmaceutically acceptable salt thereof is administered at a dose of from about 25 to about 100 mg. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the CAI, such as acetazolamide, is administered at a dosage of from about 250 mg to about 750 mg. In some embodiments, the CBD is administered at a dose of from about 0.5 to about 300 mg. In some embodiments, the CBD is administered at a dose of from about 1 to about 100 mg. In some embodiments, THC is administered at a dose of from about 0.1 to about 30 mg. In some embodiments, THC is administered at a dose of from about 1 to about 20 mg. In some embodiments, the THC is administered at a dose of from about 0.25 to about 10 mg. In some embodiments, the Dronabinol is administered at a dose of from about 1 to about 20 mg. In some embodiments, the NRI and cannabinoid are administered in a single composition. In some embodiments, the NRI, MRA, and cannabinoid are administered in a single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the invention provides a pharmaceutical composition comprising (i) a norepinephrine reuptake inhibitor (NRI) and (ii) a cannabinoid, in a pharmaceutically acceptable carrier.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the NRI is reboxetine or a pharmaceutically acceptable salt thereof. In some embodiments, the cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof. In some embodiments, the cannabinoid is CBD. In some embodiments, the cannabinoid is THC. In some embodiments, the cannabinoid is dronabinol, nabilone, or a combination thereof. In some embodiments, the cannabinoid is dronabinol. In some embodiments, the composition further comprises (iii) a muscarinic receptor antagonist (MRA). In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the composition further comprises a carbonic anhydrase inhibitor (CAI). In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the atomoxetine or pharmaceutically acceptable salt thereof is present in an amount of from about 20 to about 200 mg. In some embodiments, the atomoxetine or pharmaceutically acceptable salt thereof is present in an amount of from about 25 to about 100 mg. In some embodiments, the oxybutynin or pharmaceutically acceptable salt thereof is present in an amount of from about 1 to about 15 mg. In some embodiments, the oxybutynin or pharmaceutically acceptable salt thereof is present in an amount of from about 2 mg to about 10 mg. In some embodiments, the (R)-oxybutynin or pharmaceutically acceptable salt thereof is present in an amount of from about 0.5 to about 10 mg. In some embodiments, the (R)-oxybutynin or pharmaceutically acceptable salt thereof is present in an amount of from about 1 mg to about 5 mg. In some embodiments, the CAI, such as acetazolamide, is present in an amount of from about 250 mg to about 750 mg. In some embodiments, the CBD is present in an amount of from about 0.5 to about 300 mg. In some embodiments, the CBD is present in an amount of from about 1 to about 100 mg. In some embodiments, THC is present in an amount of from about 0.1 to about 30 mg. In some embodiments, THC is present in an amount of from about 1 to about 20 mg. In some embodiments, the THC is present in an amount of from about 0.25 to about 10 mg. In some embodiments, Dronabinol is present in an amount of from about 1 to about 20 mg. In some embodiments, the NRI and cannabinoid are formulated in a single composition. In some embodiments, the NRI, MRA, and cannabinoid are formulated in a single composition. In some embodiments, the single composition is an oral administration form. In some embodiments, the oral administration form is a syrup, pill, tablet, troche, capsule, or patch. In some embodiments, the composition is for use in treating a subject having a condition associated with pharyngeal airway collapse. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state. In some embodiments, the non-fully conscious state is sleep.

Another aspect of the invention provides a kit comprising (i) a norepinephrine reuptake inhibitor (NRI) and (ii) a cannabinoid, and optionally (iii) a muscarinic receptor antagonist. In some embodiments, the kit is for use in treating a subject having a condition associated with pharyngeal airway collapse.

Another aspect of the invention provides a norepinephrine reuptake inhibitor (NRI) and a cannabinoid, and optionally a muscarinic receptor antagonist, for use in treating a subject having a condition associated with pharyngeal airway collapse.

Another aspect of the invention provides a therapeutic combination of (i) a norepinephrine reuptake inhibitor (NRI) and (ii) a cannabinoid, and optionally (iii) a muscarinic receptor antagonist, for use in treating a subject having a condition associated with pharyngeal airway collapse.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

FIG. 2 is a schematic of the clinical study described in Example 3.

FIG. 3 is a bar graph of median apnea-hypopnea index (AHI4) for the different treatment groups, and at baseline, in the study of Example 3.

FIG. 4 is a bar graph of the geometric mean of hypoxic burden (HB4) for the different treatment groups, and at baseline, in the study of Example 3. Geometric mean can be used for HB data display because of its logarithmic distribution.

FIGS. 5A-C are bar graphs of mean total sleep time (TST), mean wake after sleep onset (WASO), and mean sleep onset latency for the different treatment groups, and at baseline, in the study of Example 3.

### DETAILED DESCRIPTION

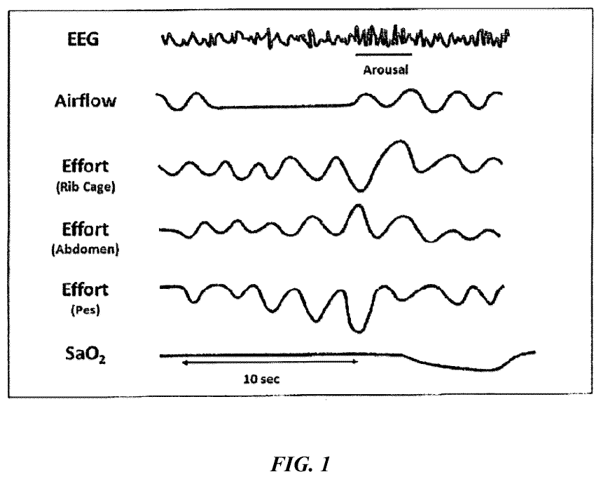

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland, W R. et al., The new AASM criteria for scoring hypopneas: Impact on the apnea hypopnea index. SLEEP 2009; 32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young, T. et al., WMJ 2009; 108:246; Peppard, P E. et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, T E. Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Hudgel, D A. et al., Chest. 1991 August; 100(2):416-21; Brownell L G. et al., N Engl J Med 1982, 307:1037-1042; Sangal R B. et al., Sleep Med. 2008 July; 9(5):506-10. Epub 2007 Sep. 27; Marshall, N S. et al. Sleep 2008 June; 31(6):824-31; Eckert, D J. et al., Clin Sci (Lond). 2011 June; 120(12); 505-14; Taranto-Montemurro, L. et al., Sleep 2017 Feb. 1; 40(2):ZSW047.

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, L. et al., The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019 May 15; 199(10):1267-1276.

The cannabinoid dronabinol has been studied in obstructive sleep apnea (OSA). See Carley, D W. et al., Pharmacotherapy of apnea by cannabimimetic enhancement, the PACE clinical trial: effects of dronabinol in obstructive sleep apnea. Sleep 2018; 41(1):ZSX184. Further study of cannabinoids for OSA and related conditions is needed.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of a norepinephrine reuptake inhibitor (NRI) and a cannabinoid, and optionally a muscarinic receptor antagonist (MRA) and/or carbonic anhydrase inhibitor (CAI), as known in the art and/or described herein, to a subject who is in need of, or who has been determined to be in need of, such treatment. In certain embodiments, the methods include administering a therapeutically effective amount of (i) atomoxetine or a pharmaceutically acceptable salt thereof, (ii) CBD or THC, and optionally (iii) oxybutynin (e.g., (R)-oxybutynin) or a pharmaceutically acceptable salt thereof, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. In some embodiments, the compositions are administered orally. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, Berge, S M. et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Atomoxetine is the generic name of the pharmaceutical substance with the chemical name (−)-N-Methyl-3-phenyl-3-(o-tolyloxy)-propylamine, and its pharmaceutical salts. Atomoxetine is the R(−)-isomer as determined by x-ray diffraction. In some embodiments, atomoxetine may be atomoxetine hydrochloride.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

“Cannabinoids” are a group of compounds including the endocannabinoids, the phytocannabinoids and those which are neither endocannabinoids nor phytocannabinoids, hereinafter “syntho-cannabinoids”. “Endocannabinoids” are endogenous cannabinoids, which are high affinity ligands of CB1 and CB2 receptors. “Phytocannabinoids” are cannabinoids that originate in nature and can be found in the cannabis plant. The phytocannabinoids can be present in an extract including a botanical drug substance, isolated, or reproduced synthetically. “Syntho-cannabinoids” are those compounds capable of interacting with the cannabinoid receptors (CB1 and/or CB2) but are not found endogenously or in the cannabis plant.

In some embodiments, the methods include administering a dose of from about 20 mg to about 200 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 40 mg. In some embodiments, the dose of atomoxetine or pharmaceutically acceptable salt thereof is about 80 mg.

In some embodiments, the methods include administering a dose of from about 0.2 mg to about 12 mg of reboxetine or a pharmaceutically acceptable salt thereof. In some embodiments, the dose of reboxetine or a pharmaceutically acceptable salt thereof is from about 1 mg to about 8 mg. In some embodiments, the dose of reboxetine or pharmaceutically acceptable salt thereof is from about 0.5 mg to about 6 mg. In some embodiments, the dose of reboxetine or pharmaceutically acceptable salt thereof is from about 2 mg to about 6 mg. In some embodiments, the dose of reboxetine or pharmaceutically acceptable salt thereof is about 4 mg. In some embodiments, the dose of reboxetine or pharmaceutically acceptable salt thereof is about 6 mg. In some embodiments, the dose of reboxetine or pharmaceutically acceptable salt thereof is about 2 mg. In some embodiments, the dose of reboxetine or pharmaceutically acceptable salt thereof is about 3 mg. In some embodiments, the reboxetine or pharmaceutically acceptable salt thereof is (S,S)-reboxetine or a pharmaceutically acceptable salt thereof.

In some embodiments, the methods include administering a dose of from about 0.5 to about 300 mg of CBD. In some embodiments, the dose of CBD or a pharmaceutically acceptable salt thereof is from about 1 to about 100 mg. In some embodiments, the dose of CBD or a pharmaceutically acceptable salt thereof is from about 1 mg to about 10 mg. In some embodiments, the dose of CBD or a pharmaceutically acceptable salt thereof is from about 10 mg to about 100 mg. In some embodiments, the CBD is administered orally. In some embodiments, the CBD is administered sublingually.

In some embodiments, the methods include administering a dose of from about 0.1 to about 30 mg of THC. In some embodiments, the methods include administering a dose of from about 1 to about 20 mg of THC. In some embodiments, the dose of THC or a pharmaceutically acceptable salt thereof is from about 0.5 to about 20 mg. In some embodiments, the dose of THC or a pharmaceutically acceptable salt thereof is from about 0.25 to about 10 mg. In some embodiments, the THC is administered orally. In some embodiments, the THC is administered sublingually.

In some embodiments, the methods include administering a dose of from about 1 mg to about 20 mg of dronabinol (e.g., daily). In some embodiments, the methods include administering a dose of from about 2.5 mg to about 10 mg of dronabinol (e.g., daily). In some embodiments, the methods include administering a dose of from about 5 mg to about 10 mg of dronabinol (e.g., daily). In some embodiments, the methods include administering a dose of about 5 mg of dronabinol (e.g., daily). In some embodiments, the methods include administering a dose of about 10 mg of dronabinol (e.g., daily).

In some embodiments, the methods include administering a dose of from about 0.25 mg to about 20 mg of nabilone (e.g., from about 0.25 mg to about 2 mg) (e.g., daily).

In methods comprising administration of oxybutynin or (R)-oxybutynin or a pharmaceutically acceptable salt thereof (or another MRA), the dose of oxybutynin or (R)-oxybutynin or pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 1 mg to about 5 mg, e.g., 2.5 mg. In some embodiments, the dose of oxybutynin or (R)-oxybutynin or pharmaceutically acceptable salt thereof is from about 1 mg to about 10 mg.

In some embodiments, the method further comprises administering a carbonic anhydrase inhibitor (CAI). In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. The CAI, such as acetazolamide, may be administered at a dose of from about 250 mg to about 750 mg.

### Pharmaceutical Compositions

Also provided herein are pharmaceutical compositions comprising a norepinephrine reuptake inhibitor (NRI) and a cannabinoid, and optionally a muscarinic receptor antagonist (MRA) and/or carbonic anhydrase inhibitor (CAI), as active ingredients. The active ingredients can be in a single composition or in separate compositions. In certain embodiments, the pharmaceutical compositions include (i) atomoxetine or a pharmaceutically acceptable salt thereof and (ii) CBD or THC, and optionally (iii) oxybutynin (e.g., (R)-oxybutynin) or a pharmaceutically acceptable salt thereof, as active ingredients.

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof. In some embodiments, the NRI is reboxetine or a pharmaceutically acceptable salt thereof.

Exemplary cannabinoids include cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA) and pharmaceutically acceptable salts thereof, or any combination thereof.

In some embodiments, the cannabinoid is CBD.

In some embodiments, the cannabinoid is THC. In some embodiments, the cannabinoid is synthetic THC. In some embodiments, the cannabinoid is a synthetic THC derivative (e.g., nabilone). In some embodiments, the cannabinoid is an enantiomerically pure form of THC (e.g., dronabinol). Dronabinol is synthetic delta-9-tetrahydrocannabinol (delta-9-THC).

Additional exemplary cannabinoids include commercially available cannabinoids such as Epidiolex® (CBD oral solution), Sativex® (nabiximols), Cesamet® (nabilone), Marinol® (dronabinol), and Acomplia® (rimonabant).

Additional exemplary cannabinoids include investigational cannabinoids such as SCI-110 (THX-110), AM-251, AM-630, HU-308, ABX-1431, RAD-011, Liquid Structure CBD, ART12.11 (CBD cocrystals), GWP-42006), CMX-020, ECP022A, Dronabinol buccal, Nabiolone controlled release, NE-1940, Olorinab, Drinabant, MDMB-FUBINACA, 5F-AB-PINACA, 5F-ADB, 5F-AMB, 5F-APINACA, AB-FUBINACA, AB-CHFUPYCA, AB-CHMINACA, AB-PINACA, ADB-CHMINACA, ADB-FUBINACA, ADSB-FUB-187, ADB-PINACA, ADBICA, APICA, Adamantyl-THPINACA, STS-135, AB-001, A-834,735, A-796,260, A-836,339, JWH-200, JWH-018, GUB-APINACA, APP-FUBINACA, MDMB-CHMICA, PX-1, PX-2, PX-3, CP-55,940, Dimethylheptylpyran, HU-210, HU-331, SR144528, WIN 55,212-2, Levonantradol, AM-2201, MEPIRAPIM, JWH-133 and Levonantradol.

Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin. In some embodiments, the muscarinic receptor antagonist is fesoterodine.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active ingredients for use in the present invention may be provided as pharmaceutically acceptable salts. For example, in some embodiments, oxybutynin is oxybutynin chloride. In some embodiments, (R)-oxybutynin is (R)-oxybutynin chloride. In some embodiments, atomoxetine is atomoxetine hydrochloride.

Pharmaceutical compositions are typically formulated to be compatible with its intended route of administration. Examples of routes of administration include systemic oral or transdermal administration, as well as sublingual administration, e.g., via tablet or spray.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

In some embodiments, the pharmaceutical composition is for use in treating a condition associated with pharyngeal airway collapse. In some embodiments, the condition is sleep apnea (e.g., OSA) or snoring (e.g., simple snoring). In certain embodiments, provided herein is a pharmaceutical composition comprising atomoxetine or a pharmaceutically acceptable salt thereof and cannabidiol or a pharmaceutically acceptable salt thereof, and optionally oxybutynin (e.g., (R)-oxybutynin) or a pharmaceutically acceptable salt thereof for use in treating sleep apnea (e.g., OSA) or snoring (e.g., simple snoring).

### Kits and Combinations

Also provided herein is a kit comprising (i) a norepinephrine reuptake inhibitor (NRI) and (ii) a cannabinoid, and optionally (iii) a muscarinic receptor antagonist. For example, the kit may comprise separate pharmaceutical compositions with each composition having a single active ingredient. The kits can be used for treating a subject having a condition associated with pharyngeal airway collapse. Various embodiments of kits will be apparent from the detailed description provided herein, including from the compositions and methods described herein.

Also provided herein is a norepinephrine reuptake inhibitor (NRI) and a cannabinoid, and optionally a muscarinic receptor antagonist, for use in treating a subject having a condition associated with pharyngeal airway collapse. Further provided herein is a therapeutic combination of (i) a norepinephrine reuptake inhibitor (NRI) and (ii) a cannabinoid, and optionally (iii) a muscarinic receptor antagonist, for use in treating a subject having a condition associated with pharyngeal airway collapse. Various embodiments of combinations and therapeutic combinations will be apparent from the detailed description provided herein, including from the compositions and methods described herein. In certain embodiments of the kits and combinations of the present invention, the NRI is atomoxetine or a pharmaceutically acceptable salt thereof, the cannabinoid is CBD or THC, and the MRA, if present, is oxybutynin (e.g., (R)-oxybutynin) or a pharmaceutically acceptable salt thereof.

### EXAMPLES

The invention is further described in the following examples, which do not limit the scope of the invention described in the claims.

### Example 1. Pilot Study

In healthy human individuals, the effect of once daily atomoxetine plus a cannabinoid (e.g., CBD or THC) on genioglossus muscle activity is measured in a pilot study.

A first group of the patients is given once daily atomoxetine plus cannabinoid. A second group of patients is given placebo. Genioglossus muscle activity (EMG_{GG}, quantified as a percentage of maximum) is measured during quiet wakefulness. Each peak EMG_{GG} of a single breath is measured and is plotted against the corresponding epiglottic pressure. In addition, EMG_{GG} is measured during stable NREM sleep.

It is expected that there will be a variable but clear reduction in EMG_{GG} activity during sleep on the placebo night and that, in contrast, when patients are administered atomoxetine plus cannabidiol, the sleep-related reduction in pharyngeal muscle activity will be partially or completely prevented.

It is expected that, compared to placebo, the tested drug will yield a much higher EMG_{GG} activity during NREM sleep. It is also expected that the drug will be effective during REM sleep for those subjects exhibiting REM sleep when administered the tested drug.

### Example 2. Crossover Study

A placebo-controlled, double-blinded, randomized, crossover trial in OSA human patients is performed. Participants receive treatment (once daily atomoxetine plus cannabinoid (e.g., CBD or THC)) or placebo in randomized order 30 minutes before sleep. The treatment is expected to reduce the apnea hypopnea index and all patients are expected to experience an improvement in OSA severity. Additional benefits expected are increased genioglossus muscle responsiveness to an increase in ventilatory drive, improved upper airway muscle activity, improved ventilation, increased oxygen levels (SaO₂), increased total sleep time and improved sleep efficiency.

### Example 3. Open-Label 4-Period Dose-Escalation Safety and Efficacy Study of Dronabinol+Atomoxetine in Participants with Obstructive Sleep Apnea

This study is designed to assess the safety and efficacy for OSA of three escalating dose combinations of atomoxetine with dronabinol, compared to baseline and to atomoxetine alone.

### Endpoints

| | Endpoints |
|---|---|
| Primary | AHI4%, ATO 80/DRO 10 vs. baseline |
| Secondary | AHI4%, ATO 80/DRO 10 vs. ATO 80 |
| | AHI4%, ATO 80/DRO 5 vs. baseline |
| | AHI4%, ATO 80/DRO 5 vs. ATO 80 |
| | HB4%, ODI4%, Total time with SaO₂ <90%, Proportion of |
| | participants with ≥50% reduction in AHI4%, HB4%, |
| | ODI4% |
| Exploratory | PGI-S |
| | PROMIS sleep impairment |
| | PROMIS sleep disturbance |
| | PROMIS fatigue |
| | AHI4%, highest dose achieved by patient (ie ATO 80/DRO |
| | 5 or ATO 80/DRO 10 vs. baseline and vs. ATO 80 |
| | AHI3 (hypopnea scored when associated with 3% O₂ |
| | desaturation) |
| | AHI3a (hypopnea scored when associated with 3% O₂ |
| | desaturation or arousal) |
| | OSA endotype endpoints (Vpassive, Vactive, Muscle |
| | Compensation, Loop Gain) |
| | PSG sleep and arousal parameters |
| Safety | Vital signs, Spontaneous adverse events, DSST, VOLT, |
| Endpoints | PVT |

Abbreviations:
AHI = apnea-hypopnea index;
ATO = atomoxetine;
DSST = digit symbol substitution test;
DRO = dronabinol;
HB = hypoxic burden;
ODI = Oxygen Desaturation Index;
OSA = obstructive sleep apnea;
PROMIS = Patient Reported Outcome Measurement Information System;
PGI-S = Patient Global Impression of Severity;
PSG = polysomnography;
PROMIS = Patient Reported Outcome Measurement Information System;
PVT = psychomotor vigilance task;
SaO₂ = oxygen saturation;
SAQLI = Sleep Apnea Quality of Life Index;
VOLT = visual object learning task

### Study Design

### Overall Design

The present study (SEED) is an open-label, 4 consecutive period dose-escalation study of combinations of atomoxetine and dronabinol in participants with moderate to severe OSA. Participants will undergo initial pre-screening to determine potential study eligibility. Participants selected for further screening should either have a previous history of OSA of a severity consistent with enrollment criteria or be at high risk (e.g. as assessed by STOP-Bang Questionnaire score). Only participants who meet all non-PSG enrollment criteria at Visit 1 are eligible for a screening PSG. On a case by case basis and with agreement of the Sponsor a PSG conducted at the site within 3 months may be used instead of the Screening PSG.

Participants who meet all enrollment criteria will receive an escalating dose of atomoxetine the first week: 3 days of atomoxetine 40 mg followed by 4 days of atomoxetine 80 mg. Participants will then receive escalating dose combinations of atomoxetine and dronabinol for the next 3 weeks. The weekly dose schedule is as follows:
Week 1: atomoxetine 40 mg×3 days, then 80 mg×4 days
Week 2: atomoxetine 40 mg/dronabinol 2.5 mg
Week 3: atomoxetine 80 mg/dronabinol 5 mg
Week 4: atomoxetine 80 mg/dronabinol 10 mg

Dose escalation will be based on safety and tolerability, as assessed at weekly clinic visits and by telephone contact with participants mid-week of each at-home dosing period. Patients who do not tolerate dose escalation will discontinue dosing.

Three on-drug PSGs will be conducted on the final night of the following dosing periods:
1st week, dosing atomoxetine 80 mg alone
3rd week, dosing atomoxetine 80 mg/dronabinol 5 mg
4th week, dosing atomoxetine 80 mg/dronabinol 10 mg

Study drug for Week 1 is dispensed to participants at Visit 2. Any unused Week 1 study drug is returned at Visit 3. Similarly, study drug for Week 2, 3, and 4 is dispensed at Visits 3, 4 and 5, and unused study drug similarly returned at the subsequent visit.

Dosing of the study treatment will occur each night at the participant's usual bedtime, both during at-home nights and Visit 3, Visit 5 and Visit 6 PSG nights. Study drug dose on PSG nights is from the supply dispensed to the participant if available, but can be provided from separate site supply.

The morning of each PSG the PGI-S, PROMIS assessments, DSST, VOLT and PVT will be administered.

An End-of-Study phone call will take place 2 weeks following the end of study drug dosing.

Participants who discontinue from the study will not be replaced. No subsequent open-label extension is planned following the study.

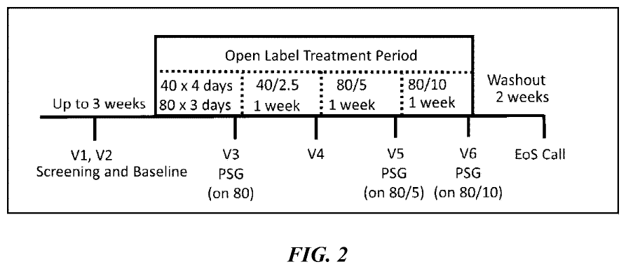

An overview of the study design is shown in FIG. 2. In FIG. 2, EoS=End of Study; PSG=polysomnography; 40=atomoxetine 40 mg alone; 80=atomoxetine 80 mg alone; 40/2.5=atomoxetine 40 mg/dronabinol 2.5 mg; 80/5=atomoxetine 80 mg/dronabinol 5 mg; 80/10=atomoxetine 80 mg/dronabinol 10 mg

### End of Study Definition

A participant is considered to have completed the study if he/she has completed all phases of the study through the last scheduled procedure shown in the Schedule of Activities (SoA) (Table 1).

The end of the study is defined as the date of the last visit of the last participant in the study or last scheduled procedure shown in the SoA for the last participant in the study globally.

### Study Population

Eligible participants will be recruited both from the existing clinic population at the study site, including databases of previous subjects who participated in other studies, and through direct advertising to the community.

Participants must be able to provide written consent and meet all the inclusion criteria and none of the exclusion criteria.

### Inclusion Criteria

1. 25 to 65 years of age, inclusive, at the Screening Visit.
2. AHI 10 to 50 (hypopneas defined by 4% oxygen desaturation)
3. ≤25% of apneas are central or mixed apneas at V2 baseline PSG
4. BMI between 18.5 and 40.0 kg/m², inclusive, at the pre-PSG visit.
5. If male and sexually active with female partner(s) of childbearing potential, participant must agree, from Study Day 1 through 1 week after the last dose of study drug, to practice the protocol specified contraception.
6. If a woman of childbearing potential (WOCBP), the participant must agree, from Study Day 1 through 1 week after the last dose of study drug, to practice the protocol specified contraception. All WOCBP must have negative result of a serum pregnancy test performed at screening.
7. If female and of non-childbearing potential, the participant must be either postmenopausal (defined as age ≥55 years with no menses for 12 or more months without an alternative medical cause) or permanently surgically sterile (bilateral oophorectomy, bilateral salpingectomy or hysterectomy).

### Exclusion Criteria

1. History of clinically significant sleep disorder other than OSA.
2. Clinically significant craniofacial malformation.
3. Clinically significant cardiac disease (e.g., rhythm disturbances, coronary artery disease or cardiac failure) or hypertension requiring more than 2 medications for control (combination medications are considered as 1 medication for this purpose).
4. Clinically significant neurological disorder, including epilepsy/convulsions.
5. History of schizophrenia, schizoaffective disorder or bipolar disorder according to Diagnostic and Statistical Manual of Mental Disorders-5 (DSM-5) or International Classification of Disease tenth edition criteria.
6. History of attempted suicide within 1 year prior to screening, or current suicidal ideation.
7. Medically unexplained positive screen for drugs of abuse or history of substance use disorder as defined in DSM-V within 24 months prior to Screening Visit.
8. A significant illness or infection requiring medical treatment in the past 30 days.
9. Clinically significant cognitive dysfunction as determined by investigator.
10. Women who are pregnant or nursing.
11. History of using devices for OSA treatment, including CPAP, oral or nasal devices, or positional devices, may enroll as long as the devices have not been used for at least 2 weeks prior to first study visit and are not used during participation in the study.
12. History of chronic oxygen therapy.
13. Use of medications from the list of disallowed concomitant medications.
14. Treatment with strong cytochrome P450 3A4 (CYP3A4) inhibitors, strong cytochrome P450 2D6 (CYP2D6) inhibitors, or monoamine oxidase inhibitors (MAOI) within 14 days of the start of treatment, or concomitant with treatment.
15. Use of another investigational agent within 30 days or 5 half-lives, whichever is longer, prior to dosing.
16. Hepatic transaminases >2× the upper limit of normal (ULN), total bilirubin >1.5× ULN (unless confirmed Gilbert syndrome), estimated glomerular filtration rate <60 ml/min.
17. PLM arousal index >20
18. <5 hours typical sleep duration.
19. ESS>18
20. Night- or shift-work sleep schedule which causes the major sleep period to be during the day.
21. Employment as a commercial driver or operator of heavy or hazardous equipment.
22. Typically smoking more than 10 cigarettes or 2 cigars per day, or inability to abstain from smoking during overnight PSG visits.
23. Unwilling to use specified contraception.
24. History of regular alcohol consumption of more than 14 standard units per week (males) or more than 7 standard units per week (females), or unwillingness to limit alcohol consumption to no greater than 2 units/day (males), 1 unit per day (females), not to be consumed within 3 hours of bedtime or on PSG nights.
25. Unwilling to limit during the study period caffeinated beverage intake (e.g., coffee, cola, tea) to 400 mg/day or less of caffeine, not to be used within 3 hours of bedtime.

### Meals and Dietary Restrictions

1. Participants should refrain from consumption of any nutrients known to modulate CYP enzyme activity (e.g., grapefruit or grapefruit juice, pomelo juice, star fruit, pomegranate, and Seville or Moro [blood] orange products) within 72 hours before the first dose of study drug and during the study.
2. Diet should be generally stable during the study, e.g., new diet programs should not be initiated.

Caffeine, Alcohol, and Tobacco
1. During the outpatient portions of the study, participants should refrain from more than 2 standard units per day of alcohol for men or 1 unit/day for women, consumed no less than 3 hours prior to bedtime. Alcohol should not be consumed on PSG nights.
2. Moderate consumption of caffeinated beverages, containing up to a total of 400 mg caffeine per day, is permitted during the study period, consumed no less than 3 hours prior to bedtime.

### Study Drug

Study drug is defined as any investigational treatment(s), marketed product(s), placebo, or medical device(s) intended to be administered to a study participant according to the study protocol.

### Study Treatment(s) Administered

One capsule of atomoxetine (week 1) or one capsule of atomoxetine and one capsule of dronabinol (weeks 2-4) is taken immediately before the participant's planned bedtime.

| Study Treatment | Atomoxetine | |
|---|---|---|
| Name: | hydrochloride | Dronabinol |
| Dosage | Capsule | Capsule |
| Formulation: | | |
| Dosage Levels: | 40 mg, 80 mg | 2.5 mg, 5 mg, 10 mg |
| Route of | Oral | Oral |
| Administration: | | |
| Dosing | 1 capsule administered | 1 capsule administered |
| Instructions: | with up to 240 mL water | with up to 240 mL water |
| Storage/Packaging/ | Store at room | Store at room |
| Labeling: | temperature, in HDPE | temperature in HDPE |
| | bottles | bottles |

### Concomitant Therapy

Concomitant therapy with the medications listed below is disallowed. For medication that is typically used as-needed for symptomatic conditions (e.g., occasional use of a sleep aid), the medication should not be used for at least one week prior to the first study PSG and for the duration of the study.
MAOIs or other drugs that affect monoamine concentrations (e.g., rasagiline) [MAOIs are contraindicated for use with atomoxetine]
Lithium
Cannabinoids
Selective Serotonin Reuptake Inhibitors (e.g., paroxetine)
Selective Norepinephrine Reuptake Inhibitors (e.g., duloxetine)
Norepinephrine Reuptake Inhibitors (e.g., reboxetine)
Alpha-1 antagonists (e.g., tamsulosin)
Tricyclic antidepressants (e.g., desipramine)
CYP2D6 inhibitors
Strong CYP3A4 inhibitors (e.g., ketoconazole)
Benzodiazepines and other anxiolytics
Opioids
Sedatives and sedative-hypnotics, including nonbenzodiazepine “Z-drugs” (zolpidem, zaleplon, eszopiclone)
Muscle relaxants
Pressor agents
Drugs with clinically significant cardiac QT-interval prolonging effects
Drugs known to lower seizure threshold (e.g., chloroquine)
Amphetamines
Antiepileptics
Antiemetics
Modafinil or armodafinil
Beta₂ agonists, (e.g., albuterol)
Antipsychotics
Sedating antihistamines
Pseudoephedrine, phenylephrine, oxymetazoline
Nicotine replacement products
Most drugs for Parkinson's, Alzheimer's, Huntington's, Amyotrophic Lateral Sclerosis, or drugs for other neurodegenerative diseases

Medications that do not have substantial effects on the central nervous system (CNS), respiration, or muscle activity are generally allowed including, but not necessarily limited to, the following drugs and drug classes:
Antihypertensives (angiotensin-converting-enzyme [ACE]/angiotensin II receptor blocker [ARB] inhibitors, calcium channel blockers, hydrochlorothiazide, etc.).
Statins
Proton pump inhibitors and histamine h₂ receptor blockers
Over-the-counter (OTC) antacids
Non-sedating antihistamines (e.g., cetirizine, loratadine)
Acetaminophen
Laxatives
Erectile dysfunction drugs
Inhaled corticosteroids (e.g., fluticasone)
Anti-diabetics
Ocular hypotensives and other ophthalmics (e.g., timolol)
Hormonal therapy (e.g., estrogen replacement or anti-estrogens) and hormonal contraceptives
Thyroid medications
Anticoagulants
Osteoporosis drugs

### Discontinuation of Study Treatment

If a clinically significant finding is identified, the Investigator or qualified designee will determine if the participant can continue in the study and if any change in participant management is needed. Any new clinically relevant finding should be reported as an adverse event (AE).

### Stopping Criteria

### 1. Individual Participant Stopping Criteria

Incidents of abuse, diversion, or misuse of the study treatment.
Incidents of clinical significance: hallucinations, amnesia, delusional thinking, delirium, manic symptoms, aggressive behavior, suicidality, homicidality, agitation, confusion, or convulsions/seizures.
Participants reporting any SAE considered possibly related or related to study treatment.
Any other AE that in the judgment of the Investigator necessitates the participant stopping to protect participant safety.

Participants discontinued from dosing will undergo end of study procedures with follow-up monitoring of any AE(s) as clinically indicated.

### Study Assessments and Procedures

Study procedures and their timing are summarized in the SoA, in Table 1.

### Polysomnography

Methods: Standard overnight PSG recording and data interpretation will be performed in accordance with the American Academy of Sleep Medicine (AASM) scoring manual. Participants will be instrumented with standard PSG electrodes. Time of lights out will be established according to the participants' habitual schedule and kept constant across the PSG study nights. The participants will be given 8 hours of time-in bed.
Participants should be actively encouraged to spend at least ⅓ of the night in the supine position and at least ⅓ of the night in the lateral position on each night of study.

### Safety Assessments

Planned time points for all safety assessments are provided in the SoA.
Safety monitoring will be guided by the established safety profiles of dronabinol and atomoxetine. Safety assessments will include physical examinations, measurement of vital signs, DSST, VOLT, PVT, monitoring and recording ofAEs, SAEs, and pregnancies, recording of study or treatment discontinuations. Effects on OSA and sleep parameters (e.g., sleep time and sleep stages) will also be monitored by PSG.

### Physical Examinations

The general physical examination at screening includes an assessment of general appearance and a review of physical systems (dermatologic, head, eyes, ears, nose, mouth/throat/neck, thyroid, lymph nodes, respiratory, cardiovascular, gastrointestinal, extremities, musculoskeletal, neurologic, and psychiatric systems). Height and weight will also be measured and recorded (with shoes removed and wearing light indoor clothing).
Investigators should pay special attention to clinical signs related to previous serious illnesses.

### Vital Signs

Assessment of vital signs (seated blood pressure, pulse rate, body temperature, respiratory rate) will be performed at the time points indicated in the SoA.
Vital signs will be measured at all visits in a seated position after 5 minutes rest and will include temperature, respiratory rate, systolic and diastolic blood pressure, and pulse. Measurements should be made in the same arm of the participant at each visit.
Systolic and diastolic blood pressure will be repeated for a total of 3 measurements, each at least 2 minutes apart.

### Electrocardiograms

A 12-lead ECG will be obtained using an ECG machine that automatically calculates the heart rate and measures the PR, QRS, and QT intervals. The ECG will be recorded in the semi-supine position after the participant has rested in this position for at least 10 minutes.

### Clinical Safety Laboratory Assessments

The Investigator must review the laboratory report and document this review. The laboratory reports must be filed with the source documents.
All protocol-required laboratory assessments must be conducted in accordance with the laboratory manual and the SoA.
If laboratory values from laboratory assessments not specified in the protocol and performed at the institution's local laboratory result in the need for a change in participant management or are considered clinically relevant by the Investigator (e.g., are considered to be an SAE or an AE or require dose modification), then the results must be recorded in the eCRF.

All AEs and SAEs will be collected from first dose of study drug until the end of the study at the timepoints specified in the SoA.

All SAEs will be recorded and reported to the Sponsor or designee within 24 hours. The Investigator will submit any updated SAE data to the Sponsor within 24 hours of it being available.

The Schedule of Activities (SoA) is shown in Table 1. The following abbreviations are used. AE=adverse event; ATO=atomoxetine; DRO=dronabinol; DSST=Digit symbol substitution test; ECG=electrocardiogram; HS=Hora Somni; PGI-S=patient global impression of severity of OSA; PSG=polysomnography; SAE=serious adverse event; SAQLI=Sleep Apnea Quality of Life Index; WOCBP=women of childbearing potential; PROMIS=Patient Reported Outcomes Measurement Information System; PVT=psychomotor vigilance test; VOLT=visual object learning task

TABLE 1
Schedule of Activities (SoA)

| | | | Dosing Period | | | | | | | | |
|---|---|---|---|---|---|---|---|---|---|---|---|
| | | Screening/ | ATO 40 3 d, | | ATO 40/ | | ATO 80/ | | ATO 80/ | | |
| | Non-PSG | Baseline | ATO 80 4 d | | DRO 2.5 | | DRO 5 | | DRO 10 | | |
| | Screening | PSG | At home | V3 | At home | | At home | | At home | PSG | EoS |
| Procedures | V1 | V2 | dosing | PSG | dosing | V4 | dosing | V5 | dosing | V6 | Call |
| Day¹ | −21 to −1 | | 1-7 ± 1 | 7 ± 1 | 8-14 ± 2 | 14 ± 2 | 15-21 ± 2 | 21 ± 2 | 21-28 ± 2 | 28 ± 2 | 42 ± 2 |
| Informed consent | X | | | | | | | | | | |
| Demography | X | | | | | | | | | | |
| Physical exam | X | | | | | | | | | | |
| Medical history | X | | | | | | | | | | |
| Pregnancy test² | X | | | | | | | | | | |
| Clinical laboratory testing | X | | | | | | | | | | |
| 12 Lead ECG | X | | | | | | | | | | |
| PSG | | X³ | | X | | | | X | | X | |
| Study drug dispense/return | | X | | X | | X | | X | | X | |
| HS study treatment⁴ | | | ↔ | ↔ | ↔ | ↔ | ↔ | ↔ | ↔ | ↔ | |
| PGI-S, PROMIS sleep | | X | | X | | | | X | | X | |
| impairment, PROMIS sleep | | | | | | | | | | | |
| disturbance, PROMIS | | | | | | | | | | | |
| fatigue, | | | | | | | | | | | |
| DSST, VOLT, PVT ⁵ | | | | | | | | | | | |
| Vital signs⁶ | X | X | | X | | X | | X | | X | |
| AE/SAE monitoring⁷ | | | ↔ | ↔ | ↔ | ↔ | ↔ | ↔ | ↔ | ↔ | ↔ |
| Prior/concomitant | | | ↔ | ↔ | ↔ | ↔ | ↔ | ↔ | ↔ | ↔ | ↔ |
| medication | | | | | | | | | | | |

¹Study days shown represent case with no use of ± window days; study days shift cumulatively if window days are used
²WOCBP only
³On a case-by-case basis with agreement of Sponsor a PSG conducted at the site within 3 months may be used instead of the Screening PSG
⁴Study medication administered at lights out; dose on PSG nights is from supply dispensed to the participant if available, but can be provided from separate site supply.
⁵ Administered at similar time on morning of each PSG visit
⁶Vital signs include seated blood pressure in triplicate, pulse, respiratory rate; vital signs on PSG nights taken morning after PSG.
⁷Site contacts participant by telephone mid-week of each at-home dosing period for safety evaluation and reminder of study activities

### Results

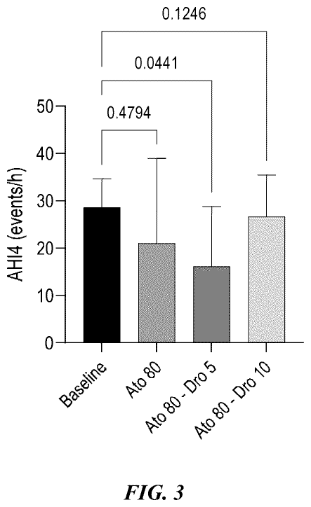

Data for the 15 patients were collected. 10 patients had PSGs in all treatment arms. Data for apnea-hypopnea index (AHI4) are shown in FIG. 3. The data show that the combination of Atomoxetine 80 mg+Dronabinol 5 mg significantly reduced AHI4 compared to baseline. Increasing the dose of dronabinol to 10 mg did not improve further the AHI. P-value was derived from Mixed effect model for repeated measures. FIG. 3 graph shows medians and interquartile range (IQR).

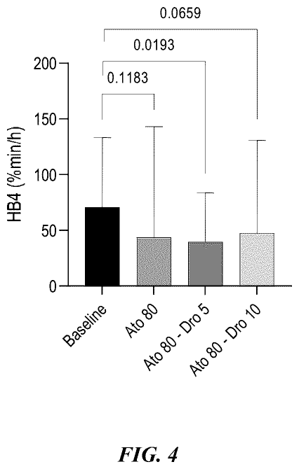

Hypoxic burden was significantly reduced on both Ato 80 mg and Ato 80 mg+Dronabinol 5 mg arms compared to baseline. The data are shown in FIG. 4. P-value was derived from Mixed effect model for repeated measures using Log(HB4+1) transformation due to known log distribution of HB. FIG. 4 graph shows geometric mean and IQR.

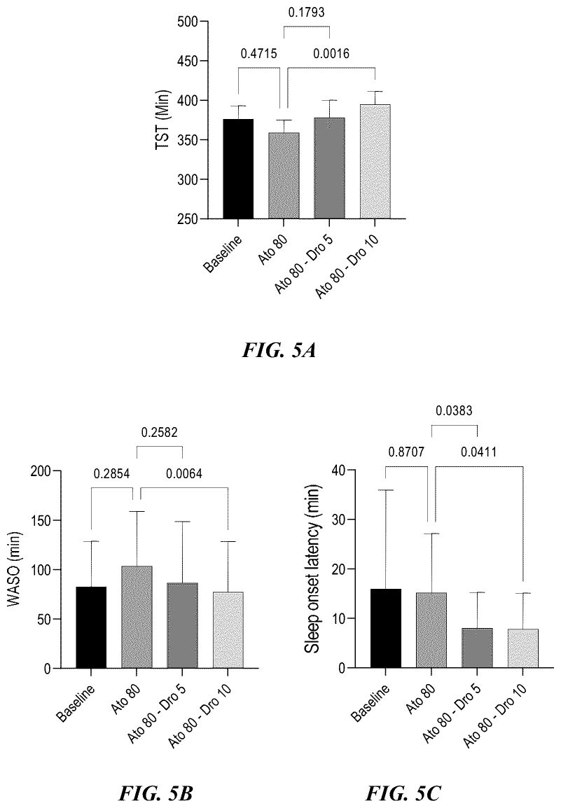

The high dose combination (Ato 80 mg-Dro 10 mg) increased total sleep time (TST) and reduced wake after sleep onset compared to Atomoxetine alone. Both doses of the combination reduced sleep onset latency compared to Atomoxetine alone. Results are shown in FIGS. 5A-C.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a norepinephrine reuptake inhibitor (NRI) and (ii) a cannabinoid.

2. The method of claim 1, wherein the NRI is a norepinephrine selective reuptake inhibitor (NSRI).

3. The method of claim 2, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

4. The method of claim 1, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

5. The method of claim 1, wherein the NRI is reboxetine or a pharmaceutically acceptable salt thereof.

6. The method of claim 1, wherein the NRI is atomoxetine or a pharmaceutically acceptable salt thereof.

7. The method of any one of claims 1-6, wherein the cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof.

8. The method of claim 7, wherein the cannabinoid is CBD.

9. The method of claim 7, wherein the cannabinoid is THC.

10. The method of claim 1, wherein the cannabinoid is dronabinol.

11. The method of any one of claims 1-10, further comprising administering to the subject (iii) a muscarinic receptor antagonist (MRA).

12. The method of claim 11, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

13. The method of any claim 11, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

14. The method of claim 11, wherein the MRA is oxybutynin or a pharmaceutically acceptable salt thereof.

15. The method of claim 14, wherein the MRA is (R)-oxybutynin or a pharmaceutically acceptable salt thereof.

16. The method of any one of claims 1-15, wherein the atomoxetine or pharmaceutically acceptable salt thereof is administered at a dose of from about 20 to about 200 mg.

17. The method of claim 16 wherein the atomoxetine or pharmaceutically acceptable salt thereof is administered at a dose of from about 25 to about 100 mg.

18. The method of any one of claims 1-17, wherein CBD is administered at a dose of from about 0.5 to about 300 mg.

19. The method of any one of claims 1-17 wherein THC is administered at a dose of from about 0.1 to about 30 mg.

20. The method of any one of claims 11-19, wherein the oxybutynin or pharmaceutically acceptable salt thereof is administered at a dose of from about 1 to about 15 mg.

21. The method of claim 20, wherein the oxybutynin or pharmaceutically acceptable salt thereof is administered at a dose of from about 2 mg to about 10 mg.

22. The method of any one of claims 11-19, wherein the (R)-oxybutynin or pharmaceutically acceptable salt thereof is administered at a dose of from about 0.5 to about 10 mg.

23. The method of claim 22, wherein the (R)-oxybutynin or pharmaceutically acceptable salt thereof is administered at a dose of from about 1 mg to about 5 mg.

24. The method of any one of claims 1-23, further comprising administering to the subject a carbonic anhydrase inhibitor (CAI).

25. The method of claim 24, wherien the CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

26. The method of claim 25, wherien the CAI is acetazolamide or a pharmaceutically acceptable salt thereof.

27. The method of any one of claims 1-26, wherein the NRI and cannabinoid are administered in a single composition.

28. The method of any one of claims 11-26, wherein the NRI, MRA, and cannabinoid are administered in a single composition.

29. The method of claim 27 or 28, wherein the single composition is an oral administration form.

30. The method of claim 29, wherein the oral administration form is a syrup, pill, tablet, troche, capsule, or patch.

31. The method of any one of claims 1-30, wherein the condition associated with pharyngeal airway collapse is sleep apnea.

32. The method of claim 31, wherein the condition associated with pharyngeal airway collapse is obstructive sleep apnea (OSA).

33. The method of any one of claims 1-30, wherein the condition associated with pharyngeal airway collapse is snoring.

34. The method of claim 33 wherein the condition associated with pharyngeal airway collapse is simple snoring.

35. The method of any one of claims 1-34, wherein the subject is in a non-fully conscious state, such as sleep.

36. A pharmaceutical composition comprising (i) a norepinephrine reuptake inhibitor (NRI) and (ii) a cannabinoid, in a pharmaceutically acceptable carrier.

37. The composition of claim 36, wherein the NRI is a norepinephrine selective reuptake inhibitor (NSRI).

38. The composition of claim 37, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

39. The composition of claim 36, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

40. The composition of claim 36, wherein the NRI is reboxetine or a pharmaceutically acceptable salt thereof.

41. The composition of claim 36, wherein the NRI is atomoxetine or a pharmaceutically acceptable salt thereof.

42. The composition of any one of claims 36-41, wherein the cannabinoid is selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA), or any combination thereof, or a pharmaceutically acceptable salt thereof.

43. The composition of claim 42, wherein the cannabinoid is CBD.

44. The composition of claim 42, wherein the cannabinoid is THC.

45. The composition of claim 36, wherein the cannabinoid is dronabinol.

46. The composition of any one of claims 36-45, further comprising (iii) a muscarinic receptor antagonist (MRA).

47. The composition of claim 46, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

48. The composition of any claim 46, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

49. The composition of claim 46, wherein the MRA is oxybutynin or a pharmaceutically acceptable salt thereof.

50. The composition of claim 49, wherein the MRA is (R)-oxybutynin or a pharmaceutically acceptable salt thereof.

51. The composition of any one of claims 36-50, wherein the atomoxetine or pharmaceutically acceptable salt thereof is present in an amount of from about 20 to about 200 mg.

52. The composition of claim 51, wherein the atomoxetine or pharmaceutically acceptable salt thereof is present in an amount of from about 25 to about 100 mg.

53. The composition of any one of claims 36-52, wherein CBD is present in an amount of from about 0.5 to about 300 mg.

54. The composition of any one of claims 36-52 wherein THC is present in an amount of from about 0.1 to about 30 mg.

55. The composition of any one of claims 46-54, wherein the oxybutynin or pharmaceutically acceptable salt thereof is present in an amount of from about 1 to about 15 mg.

56. The composition of claim 55, wherein the oxybutynin or pharmaceutically acceptable salt thereof is present in an amount of from about 2 mg to about 10 mg.

57. The composition of any one of claims 46-54, wherein the (R)-oxybutynin or pharmaceutically acceptable salt thereof is present in an amount of from about 0.5 to about 10 mg.

58. The composition of claim 57, wherein the (R)-oxybutynin or pharmaceutically acceptable salt thereof is present in an amount of from about 1 mg to about 5 mg.

59. The composition of any one of claims 36-58, further comprising a carbonic anhydrase inhibitor (CAI).

60. The composition of claim 59, wherien the CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

61. The composition of claim 60, wherien the CAI is acetazolamide or a pharmaceutically acceptable salt thereof.

62. The composition of any one of claims 36-61, wherein the NRI and cannabinoid are formulated in a single composition.

63. The composition of any one of claims 46-62, wherein the NRI, MRA, and cannabinoid are formulated in a single composition.

64. The composition of claim 62 or 63, wherein the single composition is an oral administration form.

65. The composition of claim 64, wherein the oral administration form is a syrup, pill, tablet, troche, capsule, or patch.

66. The composition of any one of claims 36-65, for use in treating a subject having a condition associated with pharyngeal airway collapse.

67. The composition for use of claim 66, wherein the condition associated with pharyngeal airway collapse is sleep apnea.

68. The composition for use of claim 67, wherein the condition associated with pharyngeal airway collapse is obstructive sleep apnea (OSA).

69. The composition for use of claim 66, wherein the condition associated with pharyngeal airway collapse is snoring.

70. The composition for use of claim 69, wherein the condition associated with pharyngeal airway collapse is simple snoring.

71. The composition for use of any one of claims 66-70, wherein the subject is in a non-fully conscious state, such as sleep.

72. A kit comprising (i) a norepinephrine reuptake inhibitor (NRI) and (ii) a cannabinoid, and optionally (iii) a muscarinic receptor antagonist (MRA) and/or carbonic anhydrase inhibitor (CAI).

73. The kit of claim 72, for use in treating a subject having a condition associated with pharyngeal airway collapse.

74. A norepinephrine reuptake inhibitor (NRI) and a cannabinoid, and optionally a muscarinic receptor antagonist (MRA) and/or carbonic anhydrase inhibitor (CAI), for use in treating a subject having a condition associated with pharyngeal airway collapse.

75. A therapeutic combination of (i) a norepinephrine reuptake inhibitor (NRI) and (ii) a cannabinoid, and optionally (iii) a muscarinic receptor antagonist (MRA) and/or carbonic anhydrase inhibitor (CAI), for use in treating a subject having a condition associated with pharyngeal airway collapse.
